(19) United States
(12) Patent Application Publication
(10) Pub. No.: US 2024/0011999 A1
(43) Pub. Date: Jan. 11, 2024

(54) METHODS FOR FINE NEEDLE SAMPLE ANALYSIS VIA MASS SPECTROMETRY

(71) Applicant: BOARD OF REGENTS, THE UNIVERSITY OF TEXAS SYSTEM, Austin, TX (US)

(72) Inventors: Livia Schiavinato EBERLIN, Austin, TX (US); Erik CRESSMAN, Houston, TX (US); Alena V. BENSUSSAN, Austin, TX (US); John LIN, Houston, TX (US); Chunxiao GUO, Houston, TX (US); Savitri KRISHNAMURTHY, Houston, TX (US)

(21) Appl. No.: 18/023,483

(22) PCT Filed: Aug. 27, 2021
(86) PCT No.: PCT/US2021/047863

§ 371 Date: Feb. 27, 2023

Related U.S. Application Data
(60) Provisional application No. 63/071,773, filed on Aug. 28, 2020.

(51) Int. Cl. G01N 33/68; G16H 50/20

(52) U.S. Cl. CPC G01N 33/6848; G16H 50/20

(57) ABSTRACT

Methods for detecting lung cancer cells, or lung cancer subtypes, by measuring levels of metabolites are provided. Methods of treating identified cancers are likewise provided

### PRIORITY CLAIM

This application claims benefit of priority to U.S. Provisional Application Ser. No. 63/071,773, filed Aug. 28, 2020, the entire contents of which are hereby incorporated by reference.

### BACKGROUND

### 1. Field

The present disclosure relates generally to the field of molecular biology, organic chemistry and oncology. More particularly, it concerns molecular profiles for cancer.

### 2. Description of Related Art

Lung cancer is the second most common cancer with over 100,000 cases diagnosed each year (1). Within its various types, non-small cell lung cancer (NSCLC) accounts for approximately 85% of all lung cancers affecting both non-smokers and smokers (2). Despite improvements in early cancer detection (3), the majority of patients with lung cancer are diagnosed at advanced stages of the disease when surgical resection is no longer curative (4, 5). Thus, management of patients with lung cancer is often focused on increasing life expectancy while maintaining quality of life, most commonly through targeted therapy, chemotherapy, radiotherapy, and immunotherapy treatment regimens. For NSCLC, targeted therapy that counteract altered biochemical pathways unique to each subtype, such as adenocarcinoma (ADC) and squamous cell carcinoma (SCC), have been increasingly explored as more efficient treatment options for patients with advanced lung cancer. Concomitantly, targeted drug treatments have made lung cancer subtyping even more crucial to properly select the drug and thus improve efficacy and patient safety (4-6). in routine clinical practice, lung cancer diagnosis and subtyping are commonly performed by image guided biopsy procedures including core needle biopsy and/or fine needle aspiration (FNA) biopsy. FNA is minimally invasive and is used to investigate lung lesions that are suspected to be malignant based on imaging findings. The aspirated material is smeared on glass slides and stained for conventional cytopathological examination or used for ancillary immunohistochemical testing. Yet, FNA diagnosis can be inconclusive in up to 30% of lung FNA biopsies due to insufficient material and/or overlap between cytological features of lung cancer subtypes, which complicates diagnosis (4, 7-9). Furthermore, the results from biopsy histologic staining and immunohistochemistry can be subjective and take several days to a week to yield a final diagnosis.

New molecular techniques capable of providing rapid and accurate diagnosis and subtyping of lung cancer from biopsy material could potentially improve management of patients with lung cancer. In the past decade, substantial strides have been made to optimize the use of lung FNAs for molecular testing using various technologies including next generation sequencing (10), mutational analysis (11), fluorescence in situ hybridization (12), and Raman spectroscopy (13-15). For example, lung FNA smears can be triaged for tumor cell macro- or microdissection, nucleic acid isolation, and subsequent molecular testing e.g., epidermal growth factor receptor (EGFR) and Kirsten Rat Sarcoma (KRAS) mutation analysis, in order to diagnose lung cancer subtype and prescribe appropriate treatment. Mass spectrometry (MS) imaging techniques have been increasingly explored for cancer diagnosis because they enable direct and untargeted analysis of tissues with high chemical specificity and analytical sensitivity (16, 17).

Several research groups have explored the use of MS imaging for lung cancer tissue diagnosis (18-23). Matrix assisted laser desorption ionization (MALDI)-MS imaging has been used to analyze the protein profiles of human lung ADC and SCC tissue sections (n=1.62), enabling discrimination between subtypes using statistical analysis with an area under the curve value of 0.885 in a validation set (19). More recently, MALDI imaging was used to analyze mock FNA samples and five clinical INA samples (23). Air flow-assisted desorption electrospray ionization-MS imaging has been used to distinguish normal and lung cancerous tissue, ADC and SCC subtypes, as well as epidermal growth factor receptor positive and wild-type ADC tissues (21, 22). Data acquired from a total of 55 human cancer and adjacent normal tissue sections enabled identification of ADC and SCC subtypes with 85.2% and 82.1% accuracies, respectively, using cross validation in a training set (22). While these results are promising, there has been limited effort evaluating the performance of MS imaging methods in clinical FNA samples. Thus, there is an unmet need for the use of desorption electrospray ionization (DESI) MS and statistical classification to diagnose and subtype (ADC and SCC) lung cancer tissue sections and FNA biopsies.

### SUMMARY

in certain embodiments, the present disclosure provides methods classification of molecular profiles of ADC and SCC lung cancer subtypes from human lung tissue and fine-needle aspiration biopsies. In a first embodiment, there is provided method of detecting cancer cells in a lung cancer sample comprising performing desorption electrospray ionization mass spectrometry imaging (DESI-MSI) on the lung cancer sample using a restricted mass range in negative ion mode to obtain a molecular profile; and applying a statistical algorithm on the molecular profile to detect the presence of cancer cells.

In some aspects, the method is further defined as an ex vivo method. In certain aspects, the method is further defined as a method for detecting lung cancer in a subject.

In certain aspects, the lung cancer is non-small cell lung cancer (NSCLC). In particular aspects, the DESI-MSI is 2D DESI-MSI. In specific aspects, the restricted mass range is m/z 500-1500. In some aspects, the molecular profile comprises metabolites, fatty acids, and lipids. In particular aspects, the molecular profile comprises glycerophosphoglycerols (PG), glycerophosphoinositols (PI), glycerophosphoserines (PS), glycerophosphoethanolamines (PE), and/or fatty acids (FA).

In some aspects, the lung cancer sample is a tissue sample. In further aspects, the method further comprises obtaining the tissue sample from a subject. In certain aspects, the method further comprises obtaining a reference profile and detecting the presence of cancer cells by comparing the profile from the sample to a reference profile. In some aspects, the reference profile is obtained from the same subject. In certain aspects, the reference profile is obtained from a different subject. In particular aspects, the lung cancer sample is a fine-needle aspiration (FNA) biopsy sample.

In particular aspects, the statistical algorithm differentiates normal lung tissue and lung cancer tissue. In some aspects, further defined as a method for classifying a lung cancer, wherein the statistical algorithm further differentiates lung cancer subtypes. In certain aspects, the lung cancer subtypes are adenocarcinoma (ADC), squamous cell carcinoma (SCC), sarcomatoid carcinoma, large cell carcinoma (LCC), and/or adenosquamous carcinoma. In some aspects, the lung cancer subtypes are ADC and SCC.

In specific aspects, the statistical algorithm is further defined as a Lasso logistic regression algorithm. In some aspects, the Lasso logistic regression algorithm uses a two-class classifier. For example, the two-class classifier is a sequential two-class classifier. In particular aspects, the sequential two-class classifier comprises a first Lasso model to differentiate normal lung tissue and lung cancer tissue molecular profiles and a second Lasso model to differentiate

ADC tissue and SCC tissue. In some aspects, the first Lasso model comprises at least 3, 4, 5, 6, 7, 8, or 9 features provided in Table 3. In certain aspects, the first Lasso model comprises at least 10 of the features provides in Table 3. In specific aspects, the first Lasso model 20, 30, 50, or 59 of the features provided in Table 3.

In some aspects, detecting cancer cells comprises detecting a high relative

abundance of PI 38:4 at m/z 885.549, PI 36:1 at m/z 863.565, PI 34:1 at m/z 835.534, CL 72:7 at 724.988, CL 74:7 at m/z 738.507, Cer d34:1 at 572.481, Cer d38.1 at m/z 656.575, Cer d42.1 at m/z 684.607, CL 72:6 at Ink 725,492, CL 74:7 at 738.507, PG 34:2 at 745.502, PG 38:4 at 797.533, and/or PI 36:4 at m/z 857.519. In particular aspects, detecting cancer cells comprises detecting a high relative abundance of PI 38:4 at m/z 885.549, PI 36:1 at ink 863.565, PI 34:1 at m/z 835.534, CL 72:7 at 724.988, CL 74:7 at m/z 738.507, Cer d34:1 at 572.481, Cer d38.1 at m/z 656.575. Cer d42,1 at m/z 684.607, CL 72:6 at m/z 725.492, CL 74:7 at 738.507, PG 34:2 at 745.502, PG 38:4 at 797.533, and/or P136:4 at ink 857.519. In some aspects, detecting cancer cells comprises detecting a low relative abundance of DG 38:4 at 680.512, PG 34:1 at m/z 747.518, PG 34:1 [i2] at 748.523, PG 34:1 [i3] at 749.52.9, PE P-38:4 at 750.544, PG 36:3 at m/z 771.518, PG 36:2 at m/z 773.534, PG 36:1 at m/z 775.548, PS 36:1 [ii] at 789.548, PS 38:4 at 810.526, and/or PI 38:5 at 883.535.

In certain aspects, the second Lasso model comprises at least 3, 4, 5, 6.7, 8, or 9 features provided in Table 4. In some aspects, the second Lasso model comprises at least 10 of the features provides in Table 4, In particular aspects, the second Lasso model comprises 11, 12, 13, 14, 15, 16, 17, 18, or 19 of the features provided in Table 4.

In some aspects, the statistical algorithm comprises total ion count (TIC) normalization, 10-fold cross validation, and/or a binning data method. In certain aspects, the statistical algorithm comprises total ion count (TIC) normalization, 10-fold cross validation, and a binning data method. In particular aspects, the statistical algorithm does not use a three-class classifier, median normalization, and/or a hierarchical clustering method.

In certain aspects, the three-class classifier simultaneously differentiates normal lung tissue, ADC, and SCC molecular profiles. In some aspects, the method is automated. In specific aspects, the method is performed in less than one hour,

In additional aspects, the method further comprises administering at least a first anticancer therapy to a subject identified to have a lung cancer. In some aspects, the anticancer therapy comprises radiation, immunotherapy, surgery or chemotherapy therapy.

A further embodiment provides a method of treating a subject comprising selecting a patient determined to have a lung cancer in accordance with the present methods; and administering at least a first anticancer therapy to the subject. In some aspects, the anticancer therapy comprises radiation, immunotherapy, surgery or chemotherapy therapy. For example, the therapy is targeting therapy to treat angiogenesis, such as Bevacizumab (Avastin) or Ramucirumab (Cyramza). In some aspects, the therapy comprises a target EGFR agent, such as Erlotinib (Tarceva), Afatinib (Gilotrif), Getfitinib (Iressa), Osimertinib (Targrisso), Dacornitinib (Vizimpro), or Necitumumab (Portrazza). The therapy may comprise ALK targeted therapy, such as Crizotinib Ceritinib (Zykadia), Alectinib (Alecensa),

Brigatinib (Alunbrig), Lorlatinib (Lorbrena), or Entrectinib (Roxlytrek). The therapy may comprise a BRAE targeted therapy, such as Dabrafenib (Tafinlar) or Trametinib (Mekinist). The therapy may be Selpercatinib (Retevmo), Capmatinib (Tabrecta), or Larotrectinib (Vitrakvi). The therapy may be a chemotherapy, such as Cisplatin, Carboplatin, Paclitaxel (Taxol), Albumin-bound paclitaxel (Abraxane), Docetaxel (Taxotere), Gemcitabine (Genzar),

Vinorelbine (Navelbine), Etoposi de (VP-16), or Pemetrexed (Alimta).

Another embodiment provides a method comprising obtaining a molecular profile from an FNA biopsy sample using 2D DESI-MSI and applying a Lasso algorithm with a sequential two-class classifier to obtain said molecular profile.

In some aspects, the 2D DESI-MSI is performed using a restricted mass range. In certain aspects, the method is further defined an ex vivo method. In particular aspects, the Lasso algorithm with a sequential two-class classifier comprises a first Lasso model to differentiate normal lung tissue and lung cancer tissue molecular profiles and a second Lasso model to differentiate ADC tissue and SCC tissue. In specific aspects, the first Lasso model comprises at least 3, 4, 5, 6, 7, 8, or 9 features provided in Table 3. In some aspects, the first Lasso model comprises at least 10 of the features provides in Table 3. In certain aspects, the first Lasso model 20, 30, 40, 50, or 59 of the features provided in Table 3.

In certain aspects, detecting cancer cells comprises detecting a high relative abundance of PI 38:4 at m/z 885.549, PI 36:1 at m/z 863,565, PI 34:1 at m/z 835.534, CL 72:7 at 724.988, CL 74:7 at m/z 738.507, Cer d34:1 at 572.481, Cer d38.1 at m/z 656.575, Cer d42.1 at m/z 684.607, CL 72:6 at m/z 725.492, CL 74:7 at 738.507, PG 34:2 at 745.502, PG 38:4 at 797.533, and/or PI 36:4 at m/z 857.519. In some aspects, detecting cancer cells comprises detecting a high relative abundance of PI 38:4 at m/z 885,549, PI 36:1 at m/z 863.565, PI 34:1 m/z 835.534, CL 72:7 at 724.988, CL 74:7 at m/z 738,507, Cer d34:1 at 572.481, Cer d38.1 at m/z 656.575, Cer d42.1 at m/z 684.607, CL 72:6 at m/z 725.492, CL 74:7 at 738.507, PG 34:2 at 745.502, PG 38:4 at 797.533, and/or 1?1 36:4 at m/z 857.519. In particular aspects, detecting cancer cells comprises detecting a low relative abundance of DG 38:4 at 680.512, PG 34:1 at m/z 747.518, PG 34:1 [i2] at 748.523, PG 34:1 [i3] at 749.529, PE P-38:4 at 750.544, PG 36:3 at m/z 771.518, PG 36:2 at m/z 773.534, PG 36:1 at m/z 775.548, PS 36:1 [i1] at 789.548, PS 38:4 at 810.526, and/or PI 38:5 at 883.535. In some aspects, the second Lasso model comprises at least 3, 4, 5, 6, 7, 8, or 9 features provided in Table 4. In certain aspects, the second Lasso model comprises at least 10 of the features provides in Table 4. In some aspects, the second Lasso model comprises 11, 12, 13, 14, 15, 16, 17, 18, or 19 of the features provided in Table 4. In particular aspects, the statistical algorithm comprises total ion count (TIC) normalization, 10-fold cross validation, and/or a binning data method. In some aspects, the statistical algorithm comprises total ion count (TIC) normalization, 10-fold cross validation, and a binning data method.

A further embodiment provides a tangible computer-readable medium comprising computer-readable code that, when executed by a computer, causes the computer to perform operations comprising receiving information corresponding a measurement of a molecule level in a test sample; and correlating the measured molecule level of the test sample with a reference level, to produce a molecular profile for the test sample.

In some aspects, the molecule is selected from those listed in Tables 3 or 4. In particular aspects, the measurement of a molecule level in the test sample comprise measurements of a plurality of molecules, In some aspects, the plurality of molecules are selected from those listed in Tables 3 or 4. In specific aspects, the measurement of a molecule level in the test sample comprises a measurement of an ion generated by mass spectroscopy corresponding to the molecule. In some aspects, the ion is selected from those of Tables 3 or 4.

In additional aspects, the method further comprises (c) receiving information corresponding a measurement of a molecule level at a plurality of 2D positions in a test sample. In some aspects, the information corresponding a measurement of a molecule level in a test sample comprises DSI-MSI data, In certain aspects, the information corresponding a measurement of a molecule level in a test sample comprises 2L) DESI-MSI data.

In further aspects, the method further comprises (c) analyzing the profile of the test sample to determine if the test sample is a lung cancer sample. In some aspects, further comprises (c) analyzing the profile of the test sample to determine if the test sample is a lung cancer subtype sample. In some aspects, the reference levels are stored in said tangible computer-readable medium. In certain aspects, the receiving information comprises receiving from a tangible data storage device information corresponding to the measurement of a molecule level in a test sample. In some aspects, the method further comprises computer-readable code that, when executed by a computer, causes the computer to perform one or more additional operations comprising: sending information corresponding the profile for the test sample to a tangible data storage device.

As used herein in the specification and claims, “a” or “an” may mean one or more. As used herein in the specification and claims, when used in conjunction with the word “comprising”, the words “a” or “an” may mean one or more than one. As used herein, in the specification and claim, “another” or “a further” may mean at least a second or more.

As used herein in the specification and claims, the term “about” is used to indicate that a value includes the inherent variation of error for the device, the method being employed to determine the value, or the variation that exists among the study subjects.

Other objects, features and advantages of the present disclosure will become apparent from the following detailed description. It should be understood, however, that the detailed description and the specific examples, while indicating certain embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIG. 1: Scheme of data extraction and lasso classifier workflow. *131 tissue banked samples were obtained for our study, but 58 were excluded for low tissue quality of normal, ADC, and SCC as determined by pathological evaluation. Samples with benign diagnoses such as pneumonia were also considered low tissue quality and excluded from the data set. 73 were used for method development. **70 FNAs were collected yet 54 were excluded as explained in Example 1, and 16 were used to test the method.

FIG. 2: Negative ion mode DESI mass spectra of ADC, SCC, and normal lung tissue. Each mass spectrum is an average of 3 scans. Optical images of corresponding ADC, SCC, and normal lung tissue sections that were H&E stained after DESI-MS imaging. Black dashed lines delineate regions of necrosis or normal stroma in isolated histology areas. Surrounding dark purple stained areas delineate regions of concentrated ADC or SCC, respectively. Scale bars=2 mm.

FIG. 3: Optical microscopy images of three H&E stained lung tissue sections with corresponding DESI-MS ion images. Black dotted lines delineate regions of ADC or SCC.

Red dotted lines delineate regions of normal lung cells and/or normal stroma. Blue dotted lines delineate regions of necrosis. Solid black squares indicate regions selected to show enlarged histology. (x:y) indicate the number of carbon atonis:double bonds in each lipid. Scale bar=4 mm.

FIGS. 4A-4B: Features selected by the Lasso models built from DESI-MS imaging data to differentiate A) normal lung from cancerous lung tissue and B) ADC from SCC lung cancer subtypes, respectively. Larger weights indicate significance of each feature for respective tissue types

FIGS. 5A-5D: Mass spectra, ion images, and optical images of H&E stained clinical FNA samples. (FIG. 5A) Selected negative ion mode mass spectrum of ADC cells from an FNA sample with its corresponding optical microcopy image, enlarged histology, and ion images. Each mass spectrum was averaged across 3 scans. (FIG. 5B) Selected negative ion mode mass spectrum of SCC cells from an FNA sample with its corresponding optical microcopy image, enlarged histology, and ion images. (FIG. 5C) Selected negative ion mode mass spectrum of ADC cells from an FNA sample of high cell density, with its corresponding optical microcopy image, enlarged histology, and ion images. (FIG. 5D) Selected negative ion mode mass spectrum of interfering blood within an FNA sample, with its corresponding optical microscopy image, enlarged histology, and ion image of a ceramide lipid. Solid black lines within the H&E stained image indicate regions from which the enlarged histology images were selected. Solid white lines correlate to enlarged ion image of indicated ions. Mass spectrum is an average of 3 scans. Scale bar in H&E image=4 mm. Scale bar in histology=500 μm.

FIGS. 6A-6D: Tandem MS of 4 classes of molecules seen in mass spectra of normal, ADC, and SCC lung tissue.

### DESCRIPTION OF ILLUSTRATIVE EMBODIMENTS

### I. THE PRESENT EMBODIMENTS

With advancements in personalized therapy, distinguishing between adenocarcinoma (ADC) and squamous cell carcinoma (SCC) subtypes of non-small cell lung cancers (NSCLC) is critical to patient care. Pre-operative minimally-invasive biopsy techniques, such as CT-guided fine needle aspiration (FNA), are becoming increasingly used as low-risk, rapid, and cost-effective methods for lung cancer diagnosis and NSCLC subtyping. Yet, histologic distinction of subtypes by FNA material can be challenging due to morphologic overlap between these subtypes and small amounts of procured samples. New technologies are needed to provide accurate diagnosis of NSCLC subtypes with FNA biopsy material and guide clinical management. Accordingly, in some embodiments, the present disclosure provides methods for desorption electrospray ionization mass spectrometry imaging (DESI-MSI) to diagnose and differentiate lung cancer subtypes from FNA biopsy material.

In the present studies, thousands of mass spectra were collected from 73 human tissue sections imaged by DESI-MS. Statistical classifiers were generated to first differentiate mass spectra of normal and cancerous tissues, followed by subtyping of lung cancer as ADC or SCC. Using this approach, 100% accuracy, 100% sensitivity, and 100% specificity for lung cancer diagnosis, and 73.5% accuracy for lung cancer subtyping for the training set of tissues, per-patient. On the validation set of tissues, 100% accuracy for lung cancer diagnosis and 94.1% accuracy for lung cancer subtyping were achieved. The classifiers were tested on mass spectra acquired from 16 FNA smears collected from patients undergoing interventional radiology guided FNA, yielding 100% diagnostic accuracy, and 87.5% accuracy on lung cancer subtyping per-slide. The results showed that DESI-MSI can be useful as an ancillary technique to conventional cytopathological examination for improved diagnosis and subtyping of lung NSCLC.

Specifically, a statistical method called least absolute shrinkage and selection operator (Lasso) was applied on the metabolic information obtained by DESI. The prediction of human lung tissue samples including 25 ADC, 26 SCC, and 22 normal lung showed an overall accuracy of 88.8%. With 8 lung FNA smears collected from MDACC, the classifier designed with the tissue mentioned earlier achieved 77.8% per-pixel overall accuracy, and 87.5% overall per-slide accuracy. Since most lung cancer patients are diagnosed at advanced stages of the disease, this next generation technology aims to reduce the recovery time, patient risk, and medical costs of invasive biopsy procedures and have a greater impact on lung cancer treatment. In some aspects, the entire analysis including sample handling and mass spectra analysis can be performed under one hour,

In particular aspects, the present methods comprise the detection of molecular signatures composed of hundreds of lipids and metabolites. The present methods can combine mass spectrometry with statistical models to diagnose lung lesions, such as using FNA biopsy material. FNA samples may be collected and analyzed using ambient ionization mass spectrometry methods, such as commercially available DESI, Flowprobe, as well as microarray droplet ionization NADI) technique and the MasSpec Pen. The MasSpec Pen deposits a droplet onto the sample and then transports the droplet with a variety of biological molecules into the mass spectrometer. Then, statistical analysis methods may be used to diagnose the lesion as adenocarcinoma or squamous cell carcinoma based on the mass spectrum obtained.

For example, Lasso may be performed for discriminating the FNA samples, which uses “sparse” models to generate simpler and easier interpretation than those from other linear regression methods, as they involve only a subset of important mass spectral features (m/z) that characterizes each tissue class. The results from the present methods were shown to have comparable prediction accuracy with that of pathological evaluation but the improvement in the workflow was seen with the simplified and expedited diagnosing process by applying DESI. In particular, the ambient mass spectrometry approach provides accurate diagnosis of lung FNA biopsies and decreases patient risk by diagnosing subtype with minimal biopsy material, and diagnosis time to select the most successful treatment option as soon as possible. The present methods can provide diagnosis within several minutes, which is much faster than that of the current gene expression diagnosis, which could take several day, and with a high accuracy rate.

Thus, the present methods can be used for detecting cancer cells and, in particular, lung cancer cells by detecting abnormal expression and composition of lipids and metabolites. These profiles can then be used to guide patient therapy. Thus, the methodologies and markers provided herein should provide a new avenue for accurate diagnosis and treatment for cancers, such as lung cancers.

### II. ASSAY METHODOLOGIES

In some aspects, the present disclosure provides methods of determining the presence of a tumor by identifying specific patterns of metabolites, lipids, and fatty acids, such as those listed in Tables 3 and 4. These patterns may be determined by measuring the presence of specific ions using mass spectroscopy. Some non-limiting examples of ionizations methods include chemical ionization, atmospheric-pressure chemical ionization, electron ionization, fast atom bombardment, electrospray ionization, and matrix-assisted laser desorption ionization, Additional ionization methods include inductively coupled plasma sources, photoionization, glow discharge, field desorption, thermospray, desorption/ionization on silicon, direct analysis in real time, secondary ion mass spectroscopy, spark ionization, and thermal ionization.

In particular, the present methods may be applied to an ambient ionization source or method for obtaining the mass spectral data such as extraction ambient ionization source. Extraction ambient ionization sources are methods with a solid or liquid extraction processes dynamically followed by ionization. Some non-limiting examples of extraction ambient ionization sources include air flow-assisted desorption electrospray ionization (AFADESI), direct analysis in real time (DART), desorption electrospray ionization (DESI), desorption ionization by charge exchange (DICE), electrode-assisted desorption electrospray ionization (EADESI), electrospray laser desorption ionization (ELDI), electrostatic spray ionization (ESTASI), Jet desorption electrospray ionization (JeDI), laser assisted desorption electrospray ionization (LADESI), laser desorption electrospray ionization (LDESI), matrix-assisted laser desorption electrospray ionization (MALDESI), nanospray desorption electrospray ionization (nano-DESI), or transmission mode desorption electrospray ionization (TM-DESI). In some embodiments, the ionization source used in the methods described herein is desorption electrospray ionization.

DESI is an ionization technique used to prepare a mass spectra of organic molecules or biomolecules. The ionization technique is an ambient ionization technique which uses atmospheric pressure in the open air and under ambient conditions, DESI is an ionization technique which combines two other ionization techniques: electrospray ionization as well as desorption ionization. Ionization is affected by directing electrically charged droplets at the surface that is millimeters away from the electrospray source. The electrospray mist is then pneumatically directed at the sample. Resultant droplets are desorbed and collected by the inlet into the mass spectrometer. These resultant droplets contain additional analytes which have been desorbed and ionized from the surface. These analytes travel through the air at atmospheric pressure into the mass spectrometer for determination of mass and charge. One of the hallmarks of DESI is the ability to achieve ambient ionization without substantial sample preparation.

As with many mass spectroscopy methods, ionization efficiency can be optimized by modifying the spray conditions such as the solvent sprayed, the pH, the gas flow rates, the applied voltage, and other aspects which affect ionization of the sprayed solution. In particular, the present methods contemplate the use of a. solvent or solution which is compatible with human issue. Some non-limiting examples of solvent which may be used as the ionization solvent include water, methanol, acetonitrile, dimethylformamide, an acid, or a mixture thereof. In some embodiments, the method contemplates a mixture of acetonitrile and. dimethylformamide. The amounts of acetonitrile and dimethylformamide may be varied to enhance the extraction of the analytes from the sample as well as increase the ionization and volatility of the sample. In some embodiments, the composition contains from about 5:1 (v/v) dimethylformamide:acetonitrile to about 1:5 (v/v) dimethyl-formamide:acetonitrile such as 1:1 (v/v) dimethylformamide:acetonittile,

Additionally, two useful parameters are the impact angle of the spray and the distance from the spray tip to the surface. Generally, the electrospray tip is placed from about 0,1-25 mm from the surface especially from 1-10 mm. In some embodiments, a placement from about 3-8 mm is useful for a wide range of different application such as those described herein. Additionally, varying the angle of the tip to the surface (known as the incident angle or α) may be used to optimize the ionization efficacy. In some embodiments, the incident angle may be from about 0° to about 90°. In some aspects, a poorly ionizing analytes such as a biomolecule will have a larger incident angle while better ionizing analytes such as low molecular weight biomolecules and organic compounds have smaller incident angle. Without wishing to be bound by any theory, it is believed that the differences in the incident angle results from the two different ionization mechanisms for each type of molecule. The poorly ionizing biomacromolecules may be desorbed by the droplet where multiple charges in the droplet may be transferred to the biomacromolecule. On the other hand, low molecular weight molecules may undergo charge transfer as either a proton or an electron. This charge transfer may be from a solvent ion to an analyte on the surface, from a gas phase solvent ion to an analyte on the surface, or from a gas phase solvent ion to a gas phase analyte molecule.

Additionally, the collection efficiency or the amount of desorbed analyte collected by the collector can be optimized by varying the collection distance from the inlet of the mass spectrometer and the surface as well as varying the collection angle (β). In general, the collection distance is relatively short from about 0 mm to about 5 mm. In some cases, the collection distance may be from about 0 mm to about 2 mm. Additionally, the collection angle (β) is also relatively small from about 1° to about 30° such as from 5° to 10°.

Each of these components may be individually adjusted to obtain sufficient ionization and collection efficiencies. Within the DESI source, the sample may be placed on a 3D moving stage which allows precise and individual control over the ionization distance, the collection distance, the incident angle, and the collection angle.

Finally, the mass spectrometer may use a variety of different mass analyzers. Some non-limiting examples of different mass analyzers include time-of-flight, quadrupole mass filter, ion trap such as a 3D quadrupole ion trap, cylindrical ion trap, linear quadrupole ion trap, or an orbitrap, or a fourier transform ion cyclotron resonance device.

The data obtained from the mass spectrometry, such as 2D DESI-MS, may then be analyzed using a statistical algorithm. The algorithm may be a Lasso logistic regression algorithm. In particular aspects, the Lasso algorithm uses a two-class classifier, such as a sequential two-class classifier. The sequential two-class classifier may comprise a first Lasso model to differentiate normal lung tissue and lung cancer tissue molecular profiles and a second Lasso model to differentiate ADC tissue and SCC tissue. Further, the Lasso statistical algorithm may comprise total ion count (TIC) normalization, 10-fold cross validation, and a binning data method.

### III. EXAMPLES

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1—DESI-MS Imaging of Lung Tissues

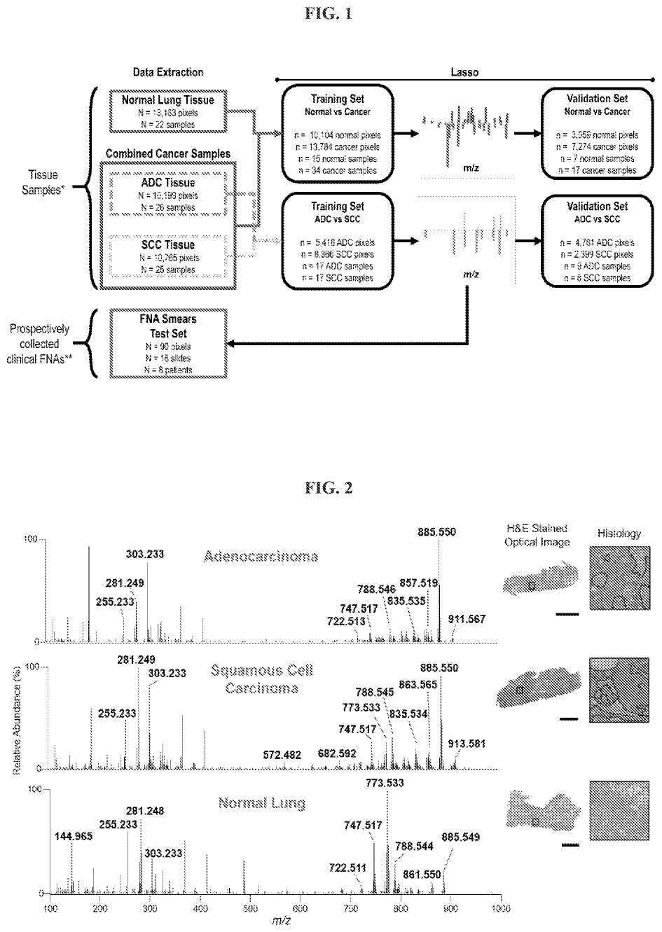

DESI-MS Imaging of Lung Tissues in the Negative Ion Mode: DESI-MSI imaging was performed on 131 lung tissue sections to acquire mass spectra of each tissue type. After pathological evaluation of tissue quality, a total of 73 lung samples, including 22 normal lung, 26 ADC, and 25 SCC samples were used to build the training and validation set, yielding a total of 34,127 mass spectra (FIG. 1). The mass spectra obtained in negative ion mode presented high relative abundances of glycerophospholipid species such as, glycerophosphoglycerois (PG), glycerophosphoinositols (PI), glycerophosphoserines (PS), glycerophosphoethanolamines (PE), and fatty acids (FA), which is typical from DESI-MSI experiments. FIG. 2 shows averaged DESI mass spectra (n=3 pixels) obtained from samples of each tissue type. Normal lung tissue mass spectra presented a molecular profile with high relative abundance of m/z 773.533, m/z 747.560 and m/z 303.233, identified as PG (36:2), PG (34:1), and FA (20:4), respectively. On the other hand, molecules such as PI (38:4) at m/z 885.550, PE (32:1) at m/z 835.534, and PS (36:1) at m/z 788.544 were detected at higher relative abundances in lung cancer tissue mass spectra when compared to normal lung tissue. While differences in the mass spectral profiles of normal and cancerous tissues could be qualitatively observed by visual inspection (FIG. 2), differences in ADC and SCC mass spectra were less prominent, requiring statistical analysis for discrimination.

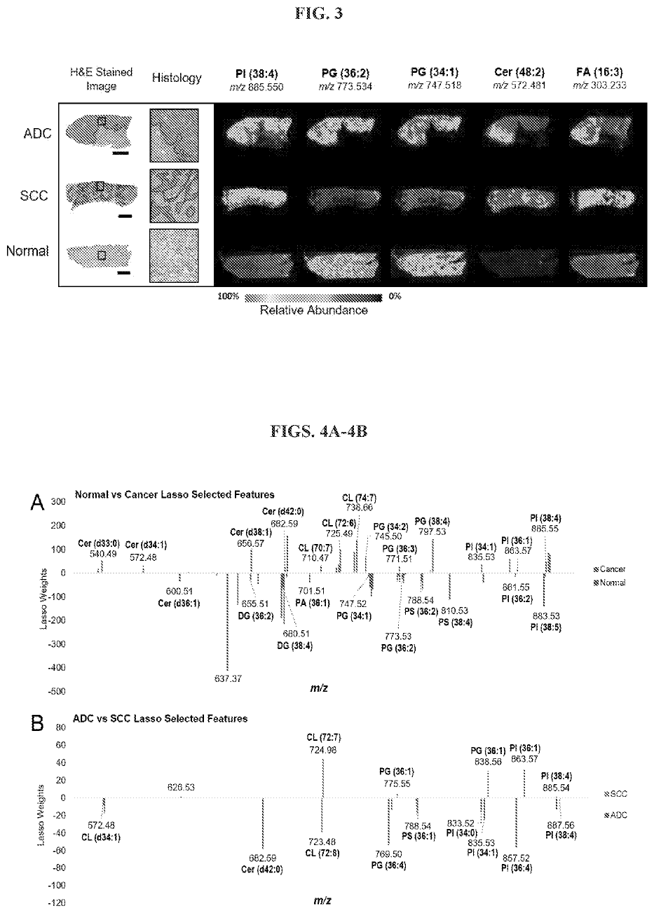

Selected DESI-MS ion images for representative tissues are shown in FIG. 3 as a visual representation of the spatial distribution of the ions selected throughout each tissue section, as well as an optical image of the stained tissue section. Histopathologic evaluation of the stained tissue sections after DESI MS imaging revealed that heterogeneity in ion distributions corresponded to tissue regions with different histologic composition, which are delineated in the optical images presented in FIG. 3 (black for cancer, red for normal, and blue for necrosis). The images obtained of several ions such as m/z 747.518 and m/z 885.550 allowed visualization of the histologic heterogeneity within normal and cancerous tissue sections. In normal lung tissue, ions at m/z 773.534 and m/z 747.518 were generally observed at a high relative abundance throughout the entire tissue sample, while lower relative abundance of these ions were seen in cancer and necrotic tissue regions. For example, visualization of a small region of normal lung in the right top corner of the SCC lung tissue shown in FIG. 3 can be visualized in the ion image of m/z 773.534 and m/z 747.518. On the other hand, m/z 885.550 was observed in high relative abundance in cancerous tissue regions, in both ADC and. SCC tissue sections. Other examples of ions observed at high relative intensity within regions of ADC and SCC tissues regions include m/z 835.534 identified as PI (34:1), m/z 863.565 identified as PI (36:1), and m/z 303.233 identified as FA (16:3). The mass spectra acquired from necrotic tissues presented overall low abundance of molecular species, except for Cer species such as m/z 572.480 identified as Cer (d34:1), and m/z 682.590 identified as Cer (d42:0), which can be seen in the ion images presented in FIG. 3. Relevant normal, ADC and SCC tissue regions demarcated by pathology were used to isolate mass spectra from surrounding stroma and necrosis and extract data for statistical analysis.

TABLE 1
Sequential classifiers performance metrics on tissue sections
of normal, adenocarcinoma (ACC), and squamous cell
carcinoma (SCC) tissues and accuracy values for validation
set of fine needle aspirate biopsies per-pixel,
per-slide, and per-patient.
Tissue Samples

| | Normal vs Cancer | | Normal vs Cancer | |
|---|---|---|---|---|
| | Per-Pixel | | Per-Patient | |
| | Training Set | Test Set | Training Set | Test Set |
| | (n = 10,104 normal, | (n = 3,059 normal, | (n = 15 normal, | (n = 7 normal, |
| | 13,784 cancer) | 7,274 cancer | 34 cancer) | 17 cancer) |
| Sensitivity (%) | 98.2 | 98.7 | 100 | 100 |
| Specificity (%) | 99.0 | 100 | 100 | 100 |
| Accuracy (%) | 98.6 | 99.1 | 100 | 100 |

| | ADC vs SCC | | ADC vs SCC | |
|---|---|---|---|---|
| | Per-Pixel | | Per-Patient | |
| | Training Set | Test Set | Training Set | Test Set |
| | (n = 5,418 | (n = 4,781 | (n = 17 | (n = 9 |
| | ADC, 8,366 SCC) | ADC, 2,399 SCC) | ADC, 17 SCC) | ADC, 8 SCC) |
| ADC Accuracy (%) | 72.6 | 68.6 | 76.5 | 88.9 |
| SCC Accuracy (%) | 72.5 | 81.3 | 70.6 | 100 |
| Overall Accuracy (%) | 72.5 | 72.8 | 73.5 | 94.1 |

| | Fine Needle Aspirate Test Set | | |
|---|---|---|---|
| | Per-Pixel | Per-Slide | Per-Patient |
| | (n = 76 | (n = 14 | (n = 7 |
| | ADC, 14 SCC) | ADC, 2 SCC) | ADC, 1 SCC) |
| ADC Accuracy (%) | 78.9 | 85.7 | 85.7 |
| SCC Accuracy (%) | 71.4 | 100 | 100 |
| Overall Accuracy (%) | 77.8 | 87.5 | 87.5 |

Statistical Classification: Two statistical models were generated in our study using Lasso logistic regression algorithm: a first model built to differentiate between normal and cancer tissues, and a second model built to subtype tissue classified as cancer as ADC versus SCC, represented in FIG. 1 (33). The Lasso selects a sparse subset of m/z values with assigned mathematical weight as a model that is predictive of tissue type using the training set of data (34). FIG. 4 shows plots of the m/z features selected by the Lasso for each model and their corresponding weights, as well as tentative identifications for a subset of the features. The normal versus lung cancer statistical model was based on 59 features, while 19 features were selected in the ADC versus SCC model. A summary of these features can be found in Tables 3 and 4. Note that while distinguishing normal from cancerous lung cells was routinely accomplished using histopathology, including this initial diagnosis step in our analysis was important for method development and necessary to identify relevant cancerous pixels for consecutive subtype differentiation.

TABLE 3
Lasso logistic regression model between normal and cancer tissues.

| | | | | |
|---|---|---|---|---|
| | Lasso | Detected | Molecular | Mass Error |
| Attribution | Weight | m/z | Formula | (ppm) |

Normal Features

| | | | | |
|---|---|---|---|---|
| Cer d36:1 | −35.4 | 600.511 | C₃₆H₇₁NO₃ | 2.3 |
| DG 34:1 | −9.5 | 629.493 | C₃₇H₇₀O₅ | −1.4 |
| unknown | −409.9 | 637.372 | — | — |
| unknown | −131.9 | 645.560 | — | — |
| DG 36:2 | −30.7 | 655.507 | C₃₉H₇₂O₅ | 0.3 |
| unknown | −49.7 | 661.557 | — | — |
| DG 38:4 | −188.0 | 679.508 | C₄₁H₂O₅ | −0.7 |
| DG 38:4 [i2] | −59.8 | 680.512 | C₄₁H₇₂O₅ | −0.3 |
| Cer 44:0 | −34.3 | 680.576 | C₄₂H₇₉NO₃ | −1.0 |
| unknown | −214.2 | 681.398 | — | — |
| unknown | −15.4 | 683.596 | — | — |
| PA 36:1 | −41.5 | 701.512 | C₃₉H₇₅O₈P | 0.6 |
| PG 34:1 | −12.6 | 747.518 | C₄₀H₇₇O₁₀P | −0.1 |
| PG 34:1 [i2] | −59.5 | 748.523 | C₄₀H₇₇O₁₀P | −2.0 |
| PG 34:0 | −97.9 | 749.529 | C₄₀H₇₇O₁₀P | −6.7 |
| PE P-38:4 | −68.6 | 750.544 | C₄₃H₇₈NO₇P | 0.5 |
| PG 36:4 | −32.8 | 769.503 | C₄₂H₇₅O₁₀P | −0.1 |
| PG 36:4 [i2] | −8.1 | 770.506 | C₄₂H₇₅O₁₀P | 0.0 |
| PG 36:3 | −36.2 | 771.518 | C₄₂H₇₇O₁₀P | 0.0 |
| PG 36:3 [i2] | −1.3 | 772.522 | C₄₂H₇₇O₁₀P | 0.0 |
| PG 36:2 | −22.1 | 773.534 | C₄₂H₇₉O₁₀P | 0.3 |
| PG 36:2 [i2] | −39.9 | 774.534 | C₄₂H₇₉O₁₀P | −0.5 |
| PG 36:1 | −7.2 | 775.548 | C₄₂H₈₁O₁₀P | 2.4 |
| PG 36:1 [12] | −2.8 | 776.553 | C₄₂H₈₁O₁₀P | −0.5 |
| PS 36:1 | −32.6 | 788.545 | C₄₂H₈₀NO₁₀P | −0.4 |
| PS 36:1 [i1] | −69.5 | 789.548 | C₄₆H₇₉O₈P | −4.3 |
| PS 38:4 | −110.9 | 810.526 | C₄₄H₇₈NO₁₀P | 3.6 |
| PE P-42:7 | −38.8 | 836.538 | C₄₇H₈₀NO₇P | −1.6 |
| PI 36:2 | −13.9 | 861.550 | C₄₅H₈₃O₁₃P | −0.2 |
| PI 36:2 [i2] | −3.3 | 862.554 | C₄₅H₈₃O₁₃P | −0.5 |
| PI 38:5 | −139.5 | 883.535 | C₄₇H₈₁O₁₃P | −0.5 |
| PI 38:5 [i2] | −12.9 | 884.538 | C₄₇H₈₁O₁₃P | −0.3 |

Cancer Features

| | | | | |
|---|---|---|---|---|
| FAHFA O-34:0 | 15.9 | 537.488 | C₃₄H₆₆O₄ | 1.3 |
| unknown | 52.1 | 540.508 | — | — |
| Cer d34:1 | 16.3 | 572.481 | C₃₄H₆₇NO₃ | 0.3 |
| DG 37:6 | 3.0 | 607.474 | C₄₀H₆₆O₅ | −1.6 |
| unknown | 0.8 | 626.536 | — | — |
| Cer d38:1 | 101.3 | 656.575 | C₄₀H₇₉NO₃ | −0.2 |
| Cer d42:2 | 21.6 | 682.592 | C₄₂H₈₁NO₃ | −1.3 |
| Cer d42:1 | 106.0 | 684.607 | C₄₂H₈₃NO₃ | −1.2 |
| Cer d42:1 | 156.6 | 684.607 | C₄₂H₈₃NO₃ | −1.2 |
| CL 70:7 | 30.8 | 710.472 | C₇₉H₁₄₀O₁₇P₂ | −1.4 |
| PE P-36:4 | 23.4 | 722.513 | C₄₁H₇₄NO₇P | −0.1 |
| CL 72:7 | 37.2 | 724.988 | C₈₁H₁₄₂O₁₇P₂ | −1.4 |
| CL 72:6 | 101.8 | 725.492 | C₈₁H₁₄₂O₁₇P₂ | −2.5 |
| unknown | 87.6 | 736.646 | — | — |
| CL 74:7 | 140.1 | 738.507 | C₈₃H₁₄₆O₁₇P₂ | −6.1 |
| PG 34:2 | 68.7 | 745.502 | C₄₀H₇₅O₁₀P | 0.3 |
| PG 36:3 | 25.9 | 771.519 | C₄₂H₇₇O₁₀P | −1.0 |
| PG 38:5 | 9.6 | 795.517 | C₄₄H₇₇O₁₀P | 1.0 |
| PG 38:4 | 142.2 | 797.533 | C₄₄H₇₉O₁₆P | 1.0 |
| PI 34:1 | 36.6 | 835.534 | C₄₃H₈₁O₁₃P | 0.1 |
| PI 36:4 | 58.6 | 857.519 | C₄₅H₇₉O₁₃P | −0.1 |
| PI 36:1 | 14.7 | 863.565 | C₄₅H₈₅O₁₃P | 0.2 |
| unknown | 47.4 | 885.540 | — | — |
| PI 38:4 | 43.6 | 885.550 | C₄₇H₈₃O₁₃P | 0.0 |
| PI 38:4 [i2] | 83.7 | 887.561 | C₄₇H₈₃O₁₃P | 0.2 |
| PI 38:4 [i3] | 75.0 | 888.567 | C₄₇H₈₃O₁₃P | −3.4 |

TABLE 4
Lasso logistic regression model between ADC and SCC.

| | | | | |
|---|---|---|---|---|
| | Lasso | Detected | Molecular | Mass Error |
| Attribution | Weight | m/z | Formula | (ppm) |

ADC Features

| | | | | |
|---|---|---|---|---|
| Cer d34:1 | −7.96 | 572.481 | C₃₄H₆₇NO₃ | 0.3 |
| Cer d34:1 [i2] | −17.04 | 573.484 | C₃₄H₆₇NO₃ | 1.2 |
| Cer d42:2 | −57.48 | 682.592 | C₄₂H₈₁NO₃ | −1.3 |
| CL 72:8 | −38.76 | 723.479 | C₈₁H₁₄₂O₁₇P₂ | −1.4 |
| unknown | −53.58 | 769.513 | — | — |
| PG 36:3 | −14.45 | 771.518 | C₄₂H₇₇O₁₀P | 0.0 |
| PS 36:1 | −2.54 | 788.545 | C₄₂H₈₀NO₁₀P | −0.4 |
| PS 36:1 [i1] | −19.29 | 789.548 | C₄₆H₇₉O₈P | −4.3 |
| PI 34:2 | −15.44 | 833.519 | C₄₃H₇₉O₁₃P | −0.6 |
| PI 34:1 | −25.33 | 835.534 | C₄₃H₈₁O₁₃P | 0.1 |
| PI 36:4 | −56.04 | 857.519 | C₄₅H₇₉O₁₃P | −0.1 |
| PI 38:4 | −12.87 | 885.550 | C₄₇H₈₃O₁₃P | 0.0 |
| PI 38:4 [i2] | −1.27 | 887.561 | C₄₇H₈₃O₁₃P | 0.2 |

SCC Features

| | | | | |
|---|---|---|---|---|
| unknown | 1.28 | 626.536 | — | — |
| CL 72:7 | 44.18 | 724.988 | C₈₁H₁₄₂O₁₇P₂ | 1.4 |
| PG 36:1 | 5.53 | 775.548 | C₄₂H₈₁O₁₀P | 2.4 |
| PS 40:4 | 30.51 | 838.556 | C₄₆H₈₂NO₁₀P | 5.1 |
| PI 36:1 | 32.80 | 863.565 | C₄₅H₈₅O₁₃P | 0.2 |
| PI 38:4 | 0.71 | 885.550 | C₄₇H₈₃O₁₃P | 0.0 |

To evaluate performance of the classifiers, predictive accuracy, sensitivity, specificity, positive predictive value (PPV) and negative predictive value (NPV) were calculated for the training and test sets of samples, both per-pixel and per-patient (Table 1). On a per-patient basis, the normal versus cancer statistical classifier yielded 100% accuracy, sensitivity, specificity, PPV and NPV for both the training and validation sets. For the ADC versus SCC statistical classifier, 73.5% of patients were correctly classified, within which 76.5% of ADC patients were correctly classified with 72.2% ADC recall and 70.6% of SCC patients were correctly classified with 75.0% SCC recall in the training set. On the validation set, high performance was achieved per-patient, with an overall accuracy of 94.1%, ADC recall of 88.9%, and 100% SCC recall. Collectively for the subtype classifier, nine samples were misclassified in the training set while one sample was misclassified in the test set.

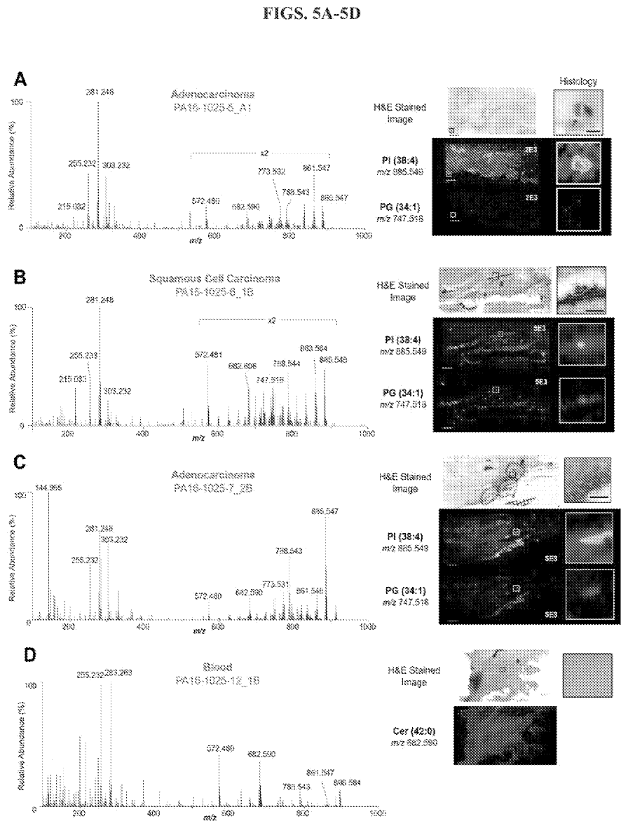

Fine-Needle Aspiration Biopsy Classification: The performance of the statistical classifiers built was next tested using tissue samples on 16 FNA slides collected from 8 lung cancer patients as an independent test set of samples. Among the 16 slides, 14 slides contained ADC cells and 2 slides contained SCC cells, per pathologic evaluation. FIG. 5 shows representative mass spectra extracted from regions containing ADC and SCC lung cells within FNA biopsies with optical images of H&E stained slide, corresponding ion images, and zoom in region to visualize histology. Generally, the mass spectra profile of FNA material were qualitatively similar to that of tissue sections in terms of the relative abundances of characteristic fatty acids and complex lipid species. Specifically, m/z 885.549, m/z 861.549, and m/z 788.545 were observed in high relative abundances in the mass spectra obtained from cancerous cells of FNA samples, as observed in the mass spectra obtained from lung cancer tissue sections (FIG. 2). An overall decrease in the relative abundance of complex lipids within m/z 500-900 when compared to FA within m/z 100 and 400 was observed in the mass spectra from FNA samples when compared to tissue sections. Further, lower total ion abundance per mass spectra was also observed in the FNA data, which can be attributed to the low cellularity of FNA samples when compared to tissues.

After pathological evaluation, data extraction was performed yielding a total of pixels or mass spectra from regions with cellular content. A varying number of cells were observed within each pixel, ranging between ˜20 cells to hundreds cells/pixel. For example, 6 pixels were extracted from FNA sample 61B from a patient diagnosed with SCC, and each pixel had ˜50 cancer cells (FIG. 5B). The remaining areas of the slide was mostly comprised. of blood (FIG. 5D). On the other hand, FNA sample 72B had 11 pixels with high cellular density (>30 c° ells/pixel) (FIG. 5C). Within the 90 pixels obtained from all samples, 76 were extracted from cells diagnosed by pathology as ADC and 14 were extracted from cells diagnosed as SCC. For information on the specific number of pixels extracted per each FNA sample and their pathologic diagnosis, Table 5.

TABLE 5
FNA test set information.

| Patient | FNA Slide | Pixels | Pathological | Lasso |
|---|---|---|---|---|
| Number | Number | Extracted | Diagnosis | Diagnosis |
| PA16-1025-1 | 1A | 3 | ADC | ADC |
| | 2A | 10 | ADC | ADC |
| PA16-1025-6 | 1B | 6 | SCC | SCC |
| | 2B | 8 | SCC | SCC |
| PA16-1025-7 | 1B | 4 | ADC | ADC |
| | 2A | 5 | ADC | ADC |
| | 2B | 11 | ADC | SCC |
| PA16-1025-10 | 1A | 3 | ADC | ADC |
| | 1B | 3 | ADC | ADC |
| PA16-1025-13 | 1B | 4 | ADC | ADC |
| PA16-1025-14 | 1B | 6 | ADC | ADC |
| | 2B | 6 | ADC | ADC |
| PA16-1025-18 | 1A | 4 | ADC | ADC |
| | 1B | 3 | ADC | SCC |
| PA16-1025-19 | 2A | 6 | ADC | ADC |
| | 2B | 8 | ADC | ADC |

Lasso models built were then applied on the extracted FNA data, and the results are reported per-pixel, per-slide, and per-patient in Table 1 First, the cancer versus normal classifier was applied, yielding 96.7% accuracy per-pixel and 100% accuracy per-sample, meaning that all slides were correctly classified as lung cancer. Considering 100% accuracy in diagnosis, the lung cancer subtyping classifier was next applied to all FNA data, yielding 84.6% overall accuracy, 81.8% ADC accuracy, 100% SCC accuracy, 100% ADC recall, and 66.7% SCC recall per-slide, On a per-patient basis, the performance metrics are similar to the per-slide metrics with 87.5% overall accuracy, 85.6% ADC accuracy with 100% ADC recall, and 100% SCC accuracy with 50% SCC recall. Generally, the results on the independent set of FNA samples were similar in performance to the prediction results obtained in the validation set of tissue samples (Table 1), which suggest robustness of the statistical classifiers.

The normal versus cancer classifier yielded high overall accuracy, sensitivity, and specificity compared to pathological evaluation of tissue sections, both per-pixel and per-sample. While the critical clinical challenge is differentiating ADC and SCC in FNA biopsies, generation of a normal versus cancer classifier was necessary to first differentiate mass spectra from normal bronchial epithelial cells, alveolar cells and alveolar macrophages that may present in FNA biopsies, prior to lung cancer subtyping. The lung cancer subtyping classifier performed well when differentiating ADC and SCC mass spectra from tissue sections, with overall accuracy of 94.1%, ADC accuracy of 88.9%, and SCC accuracy of 100% per-sample in the test set (n=17).

In conclusion, a method was developed based on metabolic information acquired using DESI-MS imaging and statistical classifiers to diagnose and subtype lung cancer and tested its performance on clinical FNA biopsies. DESI combines the advantages of MS imaging and ambient ionization MS that could be valuable in the clinical setting for direct FNA analysis, with an appealing throughput of 1 second/pixel and 2-5 hours/FNA, depending on the FNA sample size.

The high chemical sensitivity achieved in the imaging mode make this platform a powerful tool to analyze FNA biopsy material with dispersed cellular components. Combining advanced machine learning techniques with multidimensional images generated with DESI-MSI, specific regions of the heterogeneous FNA samples with relevant cellular components can be classified for diagnosis and subtyping. Further, the minimal sample preparation requirements and the non-destructive nature of DESI-MSI analysis can facilitate integration into clinical workflows. These technical features of DESI-MSI combined with robustness of statistical analysis may be appealing for clinical use, especially in cases of indeterminate and/or ambiguous pathological diagnoses (33). Collectively, the study shows that DESI-MSI holds promise as an alternative technology to current diagnostic techniques for preoperative FNA diagnosis and subtyping that could potentially improve treatment for lung cancer patients.

### Example 2—Materials and Methods

Tissue Samples: Banked frozen human tissue samples including 51 normal human lung, 40 ADC, and 40 SCC were obtained from the Cooperative Human Tissue Network and MD Anderson Cancer Center under approved IRB protocol. Normal lung specimens consist exclusively of healthy lung tissues; other benign lung tissues such as tissues diagnosed with pneumonia or other non-cancerous diseases were not included in our study. Patient demographics is presented in Table 6. Samples were stored in a −80° C. freezer. Tissue samples were sectioned at 16 μm using a CryoStar NX50 cryostat (Thermo Scientific, Waltham, MA) and thaw mounted onto semi-frost glass slides. After sectioning, the glass slides were stored in a −80° C. freezer. Prior to MS imaging, the glass slides were thawed for about 10 min. Tissue sections were stained according to hematoxylin and eosin (H&E) procedure described. A total of 73 tissue samples (22 normal lung, 25 ADC, and 26 SCC) were used in the training and validation sets.

TABLE 6
Patient demographic information for tissue samples.

| | Patient Group | | |
|---|---|---|---|
| Parameter | Normal | ADC | SCC |
| Number of Patients (N) | 22 | 25 | 26 |
| Median Age, Years | 65 | 65 | 68 |
| Age Range, Years | (5, 82) | (46, 81) | (50, 89) |
| Number of Patients by Race, | (16, 6, 0, 0) | (19, 3, 1, 2) | (20, 4, 0, 2) |
| (White, Black, Asian, Other) | | | |

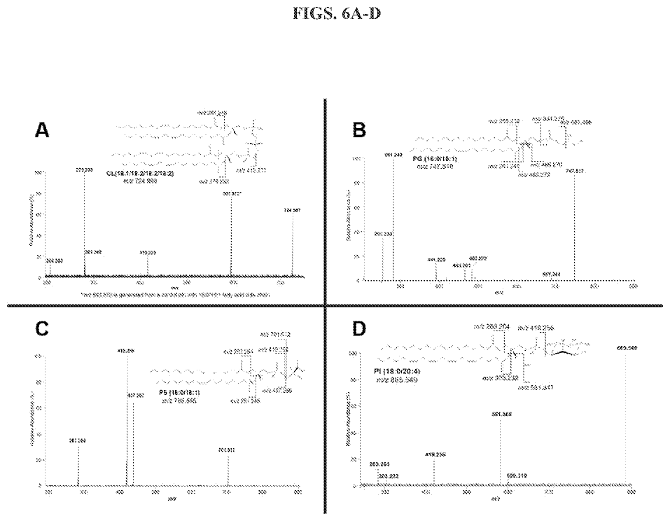

DESI-MS Imaging: A 2D Omni Spray stage (Prosolia Inc., Indianapolis, IN) with a laboratory-built sprayer was used for tissue imaging with a spatial resolution of 200 μm. DESI-MSI was performed in the negative ion mode from m/z 100-1500 with 60,000 resolving power using a LTQ-Orbitrap Elite mass spectrometer (Thermo Scientific, San Jose, CA). The histologically compatible solvent system dimethylformamide:acetonitrile (1:1) was used at 1.2 μL/min. Analysis speed was ˜1 sec/pixel or mass spectra, ˜20-50 min per tissue section, and ˜2-5 h per FNA smear. The data obtained are available on Dataverse. Tandem mass spectra of selected glycerophospholipid species detected are provided FIG. 6.

Fine Needle Aspiration Biopsy Collection and Preparation: Clinical FNA biopsies were obtained from 19 patients at MD Anderson Cancer Center under an approved IRB protocol. The FNA smears were stored at −80° C. and thawed for 5 min in ambient temperature prior to MS analysis. A total of 16 FNA slides were used in the test set. Detailed information on the FNA samples and patients is provided in FIG. 1, and Tables 6 and 7.

TABLE 7
FNA patient Information.

| | | | | | Number | |
|---|---|---|---|---|---|---|
| | | | | Number | of FNA | Final Pathological |
| Patient Number | Age | Race | Sex | of FNA | Slides | Diagnosis |
| PA16-1025-1 | 61 | W | F | 2 | 4 | Adenocarcinoma |
| PA16-1025-2 | 75 | W | F | 2 | 4 | Adenocarcinoma |
| PA16-1025-3 | 69 | W | M | 1 | 2 | Normal lung |
| PA16-1025-4 | 71 | W | F | 2 | 4 | Adenocarcinoma |
| PA16-1025-5 | 84 | W | F | 1 | 3 | Adenocarcinoma |
| PA16-1025-6 | 71 | W | F | 1 | 3 | Squamous cell |
| | | | | | | carcinoma |
| PA16-1025-7 | 49 | W | M | 2 | 4 | Adenocarcinoma |
| PA16-1025-8 | 67 | W | F | 1 | 2 | Metastatic breast |
| | | | | | | carcinoma |
| PA16-1025-9 | 56 | W | M | 2 | 4 | Squamous cell |
| | | | | | | carcinoma |
| PA16-1025-10 | 57 | W | M | 2 | 4 | Adenocarcinoma |
| PA16-1025-11 | 59 | W | M | 2 | 4 | Malignant |
| | | | | | | melanoma |
| PA16-1025-12 | 68 | W | F | 2 | 4 | Necrotizing |
| | | | | | | granulomatous |
| | | | | | | inflammation |
| PA16-1025-13 | 76 | W | F | 2 | 4 | Adenocarcinoma |
| PA16-1025-14 | 69 | W | F | 2 | 4 | Adenocarcinoma |
| PA16-1025-15 | 69 | W | F | 2 | 4 | Carcinoid Tumor |
| PA16-1025-16 | 67 | W | F | 2 | 4 | Organizing |
| | | | | | | pneumonia |
| PA16-1025-17 | 74 | W | M | 2 | 4 | Metastatic |
| | | | | | | melanoma |
| PA16-1025-18 | 61 | W | F | 2 | 4 | Adenocarcinoma |
| PA16-1025-19 | 68 | W | M | 2 | 4 | Adenocarcinoma |

Statistical Analysis: Detailed information about raw data preprocessing and statistical classifiers is provided in FIG. 1. Classifier performance was evaluated per-pixel (diagnosis for each mass spectrum/pixel), per-patient (overall diagnosis based on majority rule, more than 50% of the pixels in the sample), and per-slide (classification of the majority of pixels in a slide as the overall classification for the slide) in comparison to final pathological diagnosis.

Tissue Staining: Tissue sections analyzed by DESI-MSI were stained using standard hematoxylin and eosin (H&E) staining protocol. Pathologic evaluation was performed using light microscopy by Dr. Ruth Katz and Dr. Savitri Krishnamurthy at. MD Anderson Cancer Center (FIG. 1). Regions of high purity ADC, SCC and normal lung tissue were highlighted and annotated on each glass slide. A similar approach was taken to the FNA samples where after DESI-MSI analysis FNA slides were stained and brought for evaluation. Cell clusters of normal, ADC, and SCC were highlighted on each corresponding slide. For the study, 70 FNA slides were collected from 19 patients. A total of 12 FNA samples had fewer than 3 pixels that yielded mass spectra data and were thus excluded. In addition, 22 slides were excluded as final diagnosis revealed other types of cancer such as metastatic melanoma and breast cancer to the lung rather than primary lung cancer. Lastly, 20 slides also were eliminated as no cells were present on those slides.

Tandem Mass Spectrometry: High mass accuracy measurements were used for tentative identification of molecules detected. Collision induced dissociation was used to perform tandem mass spectrometry and confirm the identification of several abundant peaks (normalized collision energies values between 15 and 40). Collision induced dissociation was performed on a serial tissue section analyzed with DESI-MS under the same experimental conditions used for MS1 experiments. Fragmentation patterns were compared to literature reports in conjunction with data from the Lipidmaps database for identification.

Fine Needle Aspiration Collection: During this process, a patient was sedated while a suspicious lesion is repeatedly sampled for approximately 10 seconds with a 22 gauge needle. The needle was then removed and the biopsy material collected in the tip of the needle was discharged onto semi-frost glass slides. Fully frosted slides should be explored in future studies to evaluate impact on the sample and data quality obtained. A smear slide was used to spread the biopsy material along the length of each slide. These slides were then labeled and shipped in dry ice to the University of Texas at Austin. Note that the material obtained from two FNA biopsies was spread on two glass slides for the patients in this study, yielding a total of 4 FNA slides per patient. Patients with one FNA biopsy (4 of the 19 patients) yielded a total of 2 FNA slides. Two patients had one FNA biopsy spread onto three glass slides. Further, note that only 16 FNA samples were used as an independent test set from the 70 slides collected. From the remaining slides, 9 slides were excluded as they did not contain any respiratory bronchial epithelial or alveolar cells or alveolar macrophages but were mostly comprised of blood. In addition, 22 slides were excluded because final diagnoses of the tumor found in the lung for the patients consisted of metastatic cancer or other pathologies rather than primary lung cancer (8 FNA samples diagnosed as melanoma metastasis, 2 FNA samples diagnosed as breast cancer metastasis, 4 FNA samples were diagnosed as pneumonia, 4 FNA samples were diagnosed as inflammation, and 4 FNA samples were diagnosed as carcinoid tumor). The remaining 7 slides contained fewer than 3 pixels of extractable mass spectral information and were also excluded from the test set. Within those, 5 FNA smears contained very sparse cells and the SIN of biological ions for the 1-2 pixels of mass spectral data was below 3, if detected. For FNA smear 6_1A and 7_1A, higher quality (S/N>3) mass spectra was obtained but for 1 or 2 pixels in each sample, which prevented FNA classification as we used a majority rule (minimum of 3 pixels) for FNA lung cancer subtyping,.

Data Preprocessing: Xcalibur RAW files were converted into images using FireFly (v. 2.2.00) data conversion software (Prosolia, Inc. Indianapolis, IN) and then uploaded into the open source imaging software packages BioMAP version 3.8.0.4 (Novartis) or

MSiReader version 0.09 for visualization. Prior to lasso analysis, non-biological variability is minimized with preprocessing and normalization steps. Many non-biological background features were selected in the low mass range so we restricted the mass range to m/z 500-1500 to generate our statistical classifiers. Mass spectra corresponding to areas of interest highlighted by Dr. Savitri Krishnamurthy were extracted to build a database of characteristic mass spectra for all tissue types, Prior to model generation, preprocessing steps were performed such as peak reduction, binning, and normalization, to account for sample-to-sample and day-to-day variability. First, peaks that appear in fewer than 10% of pixels were eliminated from the individual mass spectra. Then, binning all peaks to 0.05 m/z was performed to account for experimental mass shifts. All of the mass spectra obtained from the pixels extracted from tissue sections and FNA smears were normalized to the total ion count prior to statistical analysis to account for variance in the total intensity due to differences in cellularity per pixel among samples. Accuracy is defined as the number of correctly classified pixels out of the total. Sensitivity is the number of correctly classified cancer pixels out of the total cancerous pixels. Specificity is the number of correctly classified normal pixels out of the total normal pixels.

Statistical Classification: Lasso logistic regression was applied to generate statistical classifiers using the publicly available glmnet package (v. 3.0-2) in the CRAM R language library (v 3.6.3). R codes for data preprocessing and statistical classifiers are available to other researchers upon request to the corresponding author. After data preprocessing, two thirds of the samples were randomly selected and used as a training set to build a statistical model using ten-fold cross validation. The remaining samples were used as a validation set to evaluate if the data were not over fit to the model generated with the training set. The samples from the training set were not included in the validation set and those from the validation set were not included in the training set. The training and validation sets were generated with mass spectra collected from tissue sections while the test set consisted of mass spectra from an independent set of FNA biopsies. Two statistical models were generated in the study: a first model was built to differentiate between normal and cancer tissues, and a second model was built to subtype tissue classified as cancer as ADC versus SCC. Each classifier was generated using the Lasso method by randomly splitting the pixels into a training set (comprising two-thirds of the data.) and a validation set (using the remaining one-third of the dataset) after normalization and data pre-processing. This workflow is represented in FIG. 1. Note that different normalization methods and mass ranges were tested to build the models, and results reported here are for models built from m/z 500-1500, which provided the best predictive performance.

Histology of Lung Tissues Analyzed by DESI-MS Imaging in the Negative Ion Mode: The samples of lung AI)C included tumors demonstrating tumor cells with vesicular nucleus, prominent nucleolus and varying grades of differentiation. Gland formation by the tumor cells was noted in well and moderately differentiated tumors and solid areas without gland formation was encountered in the poorly differentiated tumors. The majority of the cancer cases in the study were poorly differentiated ADC with solid pattern of arrangement. Tissue samples of SCC exhibited conventional histological features of squamous differentiation with sheets of tumor cells that showed different levels of differentiation. An example of a case of poorly differentiated SCC, is shown in FIG. 2, where regions of densely packed tumor cells with associated necrosis associated with adjacent normal lung parenchyma are noted. Normal lung presented typical histology of healthy lung tissue including alveoli, stroma, as well as portions of bronchi and alveoli.

Lasso Selected Features and Corresponding Weights: When evaluating the features within the Lasso model built for normal versus cancer classification, represented in FIG. 3, most of the features (11 of the 32) weighted towards normal lung tissue were identified as PG species, whereas most of the features (14 of the 26) weighted towards cancer tissue were identified as PI and Cer species (each comprising 7 of the 2.6 features, respectively). While the mathematical weights generated by the Lasso do not directly signify biological importance to each tissue type, the weights are indicative of importance in diagnosing each tissue type. In the features weighted towards cancerous tissue, for example, Cer (=42:1) at m/z 684.607 had the highest weight while PG (38:5) at m/z 795.515 received the lowest weight. In the lung cancer subtype statistical classifier, represented in FIG. 3, 13 of the 19 features selected by Lasso model were weighted towards ADC classification and six features were weighted towards SCC classification. The features selected included PI, Cer, PG, PS, and cardiolipin (CL) species. For example, Cer (42:0) at m/z 682.591, PI (36:4) at m/z 857.519, and PG (36:4) at m/z 769.502 were weighted towards ADC classification, while the highest SCC weighted features were CL (72:7) at 724.98, PI (36:1) at m/z 863.565, and PS (40:4) at m/z 838.560.

Biological Significance of Lasso Selected Features: The DESI mass spectra acquired from normal and cancerous lung tissues presented different molecular profiles, as shown in the representative data in FIG. 1. In particular, high relative abundances of PG (34:1) at m/z 747.518 and PG (36:2) at m/z 773.533 in normal lung tissues were consistently Observed in the mass spectra obtained from normal lung tissues when compared to lung cancer tissues. Both of these ions were also selected as features by the Lasso model and weighted towards normal lung tissue classification. PG species are known to play an important role in healthy lung maintenance and function, To prevent airway collapse and bacterial infection, normal lung alveolar cells secrete a pulmonary surfactant composed of 90% lipids and 10% proteins. In adults, PG species account for approximately 9.1% of the pulmonary surfactant lipid composition, which could account for the high relative abundance of specific lipid species detected.

In cancerous tissues, Cer and PI species were observed at high relative

abundances in the mass spectra obtained from cancerous lung tissue and also selected by the Lasso model with high weights for cancerous tissue classification. In particular, Cer (42:1) at m/z 684.607, and Cer (38:1) at m/z 656.575 were the features weighted highest for cancerous tissue classification. Ceramides have been implicated in cancer cell proliferation as well as biological pathways that lead to cell death through apoptosis, potentially contributing to the higher abundances detected in lung cancer tissue in our study. Considering PI species, high relative abundance of m/z 885.549 and m/z 861.550 assigned to PI (38:4) and PI (36:1), respectively, were detected in cancerous lung tissues (FIG. 1). Similar changes in PI abundances, specifically m/z 887.565 corresponding to PI (38:3) have been previously described in human lung cancer tissue by MALDI imaging, which agrees with the findings. Glycerophosphoinositols are known to be increased in NSCLC as a result of dysregulation in P13K/Akt/mTOR pathway. Nineteen features that discriminate ADC and SCC were selected by the lasso model, including several lipid species. Similarly to the cancer versus normal model, Cer and PI species were selected to differentiate ADC from SCC although no clear trends in the weights given to specific lipid classes were observed when evaluating the model.

Blood and Cellular Interference in Clinically Obtained FNA Biopsies of Lung Cancer: As expected, blood and other “interfering” cell types like inflammatory cells and alveolar macrophages were also Observed in the clinical FNA samples. The mass spectra from blood interference presented a low lipid signal and did not interfere with the mass spectra collected from the lung cancer cell clusters, as shown for a representative FNA sample in FIG. 4C. As an advantage of the proposed approach, DESI-MS imaging provides spatially accurate molecular information allowing selection of mass spectral data from lung cancer cell clusters and exclusion of mass spectra obtained from pixels containing interfering background. In addition, the histologically compatible nature of the DESI-MS imaging workflow employed here allowed H&E staining of the FNA after MS analysis, and thus unambigious correlation and matching between molecular and histologic information.

Two classifiers were generated to enable diagnosis and subtyping of lung tissues and FNA biopsies. Note that the statistical classifiers were built and validated using data obtained from tissue sections instead of FNA samples. This approach was employed to maximize the quality of the molecular data obtained from cellularly dense tissue sections and allow collection of thousands of representative mass spectra of each tissue type. Although statistical classifiers could be built and validated using the mass spectra collected from FNA biopsies, the nature of FNA biopsies including sparse cellularity and interfering biological background such as blood cells, alveolar macrophages and normal respiratory epithelial cells complicates data analysis and extraction. Given that lung cancer cells in tissue sections are in large part cytologically the same as those collected from FNA biopsies, this workflow provides an appealing approach to build robust classification models with a larger dataset Obtained from fewer samples than would be needed with FNA biopsies.

Misclassified Subtype FNA Samples: Two of the 16 FNA slides were incorrectly subtyped by our classifier compared to pathological diagnosis. These samples were vastly different in the FNA material quality. The first sample contained an unusual abundance of cells with rich molecular information that matched mass spectral quality and signal intensity seen in tissue sections, and as such, good performance was expected and the reason for misclassification is unclear. The second misclassified sample contained only 3 pixels that contained cell clusters. The mass spectra from these pixels had a low relative abundance of the features used in the statistical model, often below signal-to-noise (S/N) level of 3. Introducing S/N threshold values should be explored to evaluate the impact mass spectra quality may have on classification performance. Further, including mass spectra collected from FNA biopsies in the training set could improve FNA classification accuracy and should be explored in a larger clinical study.

All of the methods disclosed and claimed herein can be made and executed without undue experimentation in light of the present disclosure. While the compositions and methods of this disclosure have been described in terms of preferred embodiments, it will be apparent to those of skill in the art that variations may be applied to the methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### REFERENCES

The following references, to the extent that they provide exemplary procedural or other details supplementary to those set forth herein, are specifically incorporated herein by reference.
1. Siegel R L, Miller K D, Jemal A. Cancer statistics, 2019. CA Cancer J Clin 2019; 69:7-34.
2. What is lung cancer?: Types of lung cancer (Accessed Oct. 1, 2019).
3. Shen J, Todd N W, Zhang H, Yea L, Lingxiao X, Y P, et al. Plasma micromas as potential biomarkers for non-small-cell lung cancer. Laboratory investigation 2011; 91:579-87.
4. Travis W D, Rekhtman N, Riley G J, Geisinger K R, Asamura H, Brambilla E, et al. Pathologic diagnosis of advanced lung cancer based on small biopsies and cytology: A paradigm shift. J Thorac Oncol 2010; 5:411-4.
5. Rolfo C, Passiglia F, Ostrowski M, Farracho L, Ondoichova T, Dolcan A, et al. Improvement in lung cancer outcomes with targeted therapies: An update for family physicians. Journal of the American Board of Family Medicine 2015; 28:124-33.
6. Travis W D. Classification of lung cancer. Seminars in Roentgenology 2011; 46:178-86.
7. Thunnissen E, Kerr K M, Herth F J, Lantuejoul S, Papotti M, Rintoul R C, et al. The challenge of nsclc diagnosis and predictive analysis on small samples. Practical approach of a working group. Lung Cancer 2012; 76:1-18.
8. Travis W D, Brambilla E, Noguchi M, Nicholson A G, Geisinger K, Yatabe Y, et al. Diagnosis of lung cancer in small biopsies and cytology implications of the 2011 international association for the study of lung cancer/american thoracic society/european respiratory society classification. Archives of Pathology & Laboratory Medicine 2013; 137:668-84.
9. Biancosino C, Kruger M, Vollmer E, Welker L. Intraoperative fine needle aspirations—diagnosis and typing of lung cancer in small biopsies: Challenges and limitations. Diagn Pathol 2016; 11:59.
10. Kanagal-Shamanna R, Portier B P, Singh R R, Routbort M J, Aldape K D, Handal B A, et al. Next-generation sequencing-based multi-gene mutation profiling of solid tumors using fine needle aspiration samples: Promises and challenges for routine clinical diagnostics. Modern Pathology 2014; 27:314-27.
11. Smouse Cibas E S, Janne P A, Joshi V A, Lou K H, Lindeman N I. Egfr mutations are detected comparably in cytologic and surgical pathology specimens of nonsmall cell lung cancer. Cancer 2009; 117:67-72.
12. Savic S, Bubendorf L. Role of fluorescence in situ hybridization in lung cancer cytology. Acta Cytol 2012; 56:611-21.
13. Bird B. Miljkovic M S, Remiszewski S. Akalin Kon M, Diem M. Infrared spectral histopathology (shp): A novel diagnostic tool for the accurate classification of lung cancer. Lab Invest 2012; 92:1358-73.
14. Huang Z, McWilliams A, Lui H, McLean D I, Lam S, Zeng H. Near-infrared raman spectroscopy for optical diagnosis of lung cancer. Intl Cancer 2003; 107:1047-52.
15. Park J, Hwang M. Choi B, Jeong H, Jung J H, Kim H K, et al. Exosome classification by pattern analysis of surface-enhanced raman spectroscopy data for lung cancer diagnosis. Anal Chem 2017; 89:6695-701.
16. van Hove E R A, Smith D F, Heeren R M A. A concise review of mass spectrometry imaging. Journal of Chromatography A 2010; 1217:3946-54.
17. Feider C L, Krieger A, DeHoog R J, Eberlin L S. Ambient ionization mass spectrometry: Recent developments and applications. Analytical Chemistry 2019; 91:4266-90.
18. Lee G K, Lee H S, Park Y S, Lee J H, Lee S C, Lee J H, et al. Lipid maldi profile classifies non-small cell lung cancers according to the histologic type. Lung Cancer 2012; 76:197-703.
19. Marien E, Meister M, Muley T, Fieuws S, Bordel S, Derua R, et al. Non-small cell lung cancer is characterized by dramatic changes in phospholipid profiles. International Journal of Cancer 2015; 137:1539-48.
20. Kriegsmann M, Casadonte R, Kriegsmann J, Dienemann Schirmacher P, Kobarg J H, et al. Reliable entity subtyping in non-small cell lung cancer by matrix-assisted laser desorption/ionization imaging mass spectrometry on formalin-fixed paraffin-embedded tissue specimens. Molecular & Cellular Proteornics 2016; 15:3081-9.
21. Li T G, He J M, Mao X X, Bi Y, Luo Z G, Guo C G, et al. in situ biomarker discovery and label-free molecular histopathological diagnosis of lung cancer by ambient mass spectrometry imaging. Scientific Reports 2015; 5.
22. Zhang M, He J M, Li T G, Hu H X, Li X F, Xing H, et al. Accurate classification of non-small cell lung cancer (nsclc) pathology and mapping of egfr mutation spatial distribution by ambient mass spectrometry imaging. Frontiers in Oncology 2019; 9:11.
23. Amann J M, Chaurand P, Gonzalez A, Mobley J A, Massion PP. Carbone D P, Caprioli R M. Selective profiling of proteins in lung cancer cells from fine-needle aspirates by matrix-assisted laser desorption ionization time-of-flight mass spectrometry, Clin Cancer Res 2006; 12:5142-50.
24. Takats Z, Wiseman S M, Gologan B, Cooks R G. Mass spectrometry sampling under ambient conditions with desorption electrospray ionization. Science 2004; 306:471-3.
25. Ifa D R, Eberlin L S. Ambient ionization mass spectrometry for cancer diagnosis and surgical margin evaluation. Clinical Chemistry 2016; 62:111-23.
26. DeHoog R J, Zhang J L, More E, Lin J Q, Yu W D, Woody S, et al. Preoperative metabolic classification of thyroid nodules using mass spectrometry imaging of fine-needle aspiration biopsies. Proceedings of the National Academy of Sciences of the United States of America 2019; 116:21401-8.
27. Woolman M, Tata A, Bluemke E, Dara D, Ginsberg H J, Zarrine-Afsar A. An assessment of the utility of tissue smears in rapid cancer profiling with desorption electrospray ionization mass spectrometry (desi-ms). Journal of the American Society for Mass Spectrometry 2017; 28:145-53.
28. Basu S S, Regan M S, Randall E C, Abdeltnoula W M, Clark A R, Lopez B G C, et al. Rapid maldi mass spectrometry imaging for surgical pathology. Npj Precision Oncology 2019; 3.
29. Pirro V, Alfaro C M, Jarmusch A K, Hattab E M, Cohen-Gadol A A, Cooks R G.

Intraoperative assessment of tumor margins during glioma resection by desorption electrospray ionization-mass spectrometry. Proc Natl Acad Sci U S A 2017; 114:6700-5.
30. Porcari A M, Zhang J L, Garza K Y, Rodrigues-Peres R M, Lin J Q, Young J H, et al. Multicenter study using desorption-electrospray-ionization-mass-spectrometry imaging for breast-cancer diagnosis. Analytical Chemistry 2018; 90:11324-32.
31. Doria M L, MCKenzie J S, Mroz A, Phelps D L, Speller A, Rosini F, et al. Epithelial ovarian carcinoma diagnosis by desorption electrospray ionization mass spectrometry imaging. Sci Rep 2016; 6:39219.
32. Calligaris D, Caragacianu D, Liu X, Norton I, Thompson C J, Richardson A L, et al. Application of desorption electrospray ionization mass spectrometry imaging in breast cancer margin analysis. Proc Natl Acad Sci U S A 2014; 111:15184-9.
33. Adamson A S, Welch H G, Machine learning and the cancer-diagnosis problem—no gold standard. New England Journal of Medicine 2019; 381:2285-7.
34. Tibshirani R. Regression shrinkage and selection via the lasso. Journal of the Royal Statistical Society Series B-Methodological 1996; 58:267-88.
35. Eberlin L S, Ferreira C R, Dill A L, Ifa D R, Cheng L, Cooks R G. Nondestructive, histologically compatible tissue imaging by desorption electrospray ionization mass spectrometry. Chembiochem 2011; 12:2129-32.

1. A method of detecting cancer cells in a lung cancer sample comprising:
(a) performing desorption electrospray ionization mass spectrometry imaging (DESI-MSI) on the lung cancer sample using a restricted mass range in negative ion mode to obtain a molecular profile; and
(b) applying a statistical algorithm on the molecular profile to detect the presence of cancer cells.

2. (canceled)

3. (canceled)

4. The method of claim 1, wherein the lung cancer is non-small cell lung cancer (NSCLC).

5. The method of claim 1, wherein the DESI-MSI is 2D DESI-MSI.

6. The method of claim 1, wherein the restricted mass range is m/z 500-1500.

7. The method of claim 1, wherein the molecular profile comprises metabolites, fatty acids, and lipids.

8. The method of claim 1, wherein the molecular profile comprises glycerophosphoglycerols (PG), glycerophosphoinositols (PI), glycerophosphoserines (PS), glycerophosphoethanolamines (PE), and/or fatty acids (FA).

9. (canceled)

10. (canceled)

11. The method of claim 1, further comprising obtaining a reference profile and detecting the presence of cancer cells by comparing the profile from the sample to a reference profile.

12. (canceled)

13. (canceled)

14. The method of claim 1, wherein the lung cancer sample is a fine-needle aspiration (FNA) biopsy sample.

15. The method of claim 1, wherein the statistical algorithm differentiates normal lung tissue and lung cancer tissue.

16. The method of claim 15, further defined as a method for classifying a lung cancer, wherein the statistical algorithm further differentiates lung cancer subtypes.

17. The method of claim 16, wherein the lung cancer subtypes are adenocarcinoma (ADC), squamous cell carcinoma (SCC), sarcomatoid carcinoma, large cell carcinoma (LCC), and/or adenosquamous carcinoma.

18. (canceled)

19. The method of claim 1, wherein the statistical algorithm is a Lasso logistic regression algorithm. (Original) The method of claim 19, wherein the Lasso logistic regression algorithm uses a two-class classifier.

21. The method of claim 20, wherein the two-class classifier is a sequential two-class classifier.

22. The method of claim 21, wherein the sequential two-class classifier comprises a first Lasso model to differentiate normal lung tissue and lung cancer tissue molecular profiles and a second Lasso model to differentiate ADC tissue and SCC tissue.

23. The method of claim 22, wherein the first Lasso model comprises at least 3 features provided in Table 3, wherein the second Lasso model comprises at least 3 features provided in Table 4, or a combination thereof.

24. (canceled)

25. (canceled)

26. The method of claim 1, wherein detecting cancer cells comprises detecting a high relative abundance of PI 38:4 at m/z 885.549, PI 36:1 at m/z 863.565, PI 34:1 at m/z 835.534, CL 72:7 at 724.988, CL 74:7 at m/z 738.507, Cer d34:1 at 572.481, Cer d38.1 at m/z 656.575, Cer d42.1 at m/z 684.607, CL 72:6 at m/z 725.492, CL 74:7 at 738.507, PG 34:2 at 745.502, PG 38:4 at 797.533, and/or PI 36:4 at m/z 857.519.

27. (canceled)

28. The method of claim 1, wherein detecting cancer cells comprises detecting a low relative abundance of DG 38:4 at 680.512, PG 34:1 at m/z 747.518, PG 34:1 [i2] at 748.523, PG 34:1 [i3] at 749.529, PE P-38:4 at 750.544, PG 36:3 at m/z 771.518, PG 36:2 at m/z 773.534, PG 36:1 at m/z 775.548, PS 36:1 [i1] at 789.548, PS 38:4 at 810.526, and/or PI 38:5 at 883.535.

29. (canceled)

30. (canceled)

31. (canceled)

32. The method of claim 1, wherein the statistical algorithm comprises total ion count (TIC) normalization, 10-fold cross validation, and/or a binning data method.

33-36. (canceled)

37. The method of claim 1, wherein the method is performed in less than one hour.

38-70. (canceled)
